(19) United States
(12) Patent Application Publication
(10) Pub. No.: US 2025/0378553 A1
(43) Pub. Date: Dec. 11, 2025

(54) CONFOCAL AND PHOTOACOUSTIC HYBRID IMAGING DEVICE

(71) Applicant: Skincure Oncology LLC, Burr Ridge, IL (US)

(72) Inventors: Kalman Fishman, Boca Raton, FL (US); Steven Scott, Burr Ridge, IL (US)

(21) Appl. No.: 19/093,085

(22) Filed: Mar. 27, 2025

Related U.S. Application Data
(60) Provisional application No. 63/572,685, filed on Apr. 1, 2024.

(51) Int. Cl. G06T 7/00; A61N 5/06; A61N 5/067; G06T 7/55; G06T 7/62; G06T 17/00; G06V 20/64; G06V 40/10; G16H 20/40

(52) U.S. Cl. CPC G06T 7/0012; A61N 5/067; G06T 7/55; G06T 7/62; G06T 17/00; G06V 20/64; G16H 20/40; A61N 2005/0626; G06T 2207/10056; G06T 2207/30088; G06T 2207/30096; G06T 2207/30196; G06T 2207/30204; G06T 2210/41; G06V 40/10; G06V 2201/032

(57) ABSTRACT

A radiotherapy system for treatment of skin includes a confocal microscopy imaging component and a photoacoustic microscopy imaging component to capture images of the skin of a patient at more than one depth. A processor combines images to produce a fused model for the region of interest, generates a plan for radiotherapy treatment based on the fused model, and controls the radiotherapy component for carrying out the radiotherapy treatment.

### CROSS-REFERENCE TO RELATED APPLICATIONS

This application claims the benefit of, and priority to, U.S. Provisional Application No. 63/572,685, titled “Confocal and Photoacoustic Hybrid Imaging Device,” filed on Apr. 1, 2024, the disclosures of which are incorporated by reference herein in their entirety.

### FIELD OF THE INVENTION

The present disclosure relates generally to detecting skin conditions such as skin cancers.

### BACKGROUND OF THE INVENTION

Skin cancers are typically viewed as a simpler problem than other types of cancers. Since skin cancers often appear directly on the surface of the skin, skin cancers are thought of as largely a two-dimensional problem as opposed to a three-dimensional problem as in other types of cancer. In view of this, unlike other types of cancers, skin cancers are frequently treated without the use of advanced imaging equipment.

Practitioners frequently begin evaluating skin cancers by directly observing the lesion on the surface of the skin, and performing a biopsy of the lesion. Based on the result of the biopsy, the practitioner may estimate a margin and depth of skin that must be excised to remove the lesion by surgical or other means. Unfortunately, this approach to cutaneous oncology can lead to errors with regard to optimal treatment. Therefore, like other types of cancers, skin cancers can be approached as a three-dimensional problem, and the evaluation of the lesion may be performed using accurate imaging because accurate imaging is an important component of treating cancer in the field of radiation oncology. This suboptimal treatment is in part due to the fact that skin cancer is not approached and quantified like other cancers-even though it is the most prevalent.

### SUMMARY

Aspects of the subject technology relate to a radiotherapy method and system for treatment of skin. The radiotherapy method includes acquiring first image data for a region of interest in the skin of a patient using a confocal microscopy; acquiring second image data for the region of interest in the skin of the patient using a photoacoustic microscopy; combining the first image data and the second image data to produce a fused model for the region of interest in the skin of the patient; generating a plan for radiotherapy treatment of the region of interest based on the fused model; and applying the radiotherapy treatment according to the generated plan. The radiotherapy system includes a radiotherapy component comprising a radiation source configured for radiation therapy; a first imaging component comprising a confocal microscopy configured to capture one or more optical images of a region of interest in a skin of a patient for a first depth; a second imaging component comprising a photoacoustic microscopy configured to capture one or more optical images of a region of interest in the skin of the patient for a second depth deeper than the first depth; and a processor configured to acquire, using the first imaging component, a first optical image of the region of interest for the first depth; acquire, using the second imaging component, a second optical image of the region of interest for the second depth; combine the first optical image and the second optical image to produce a fused model for the region of interest; generate a plan for radiotherapy treatment of the region of interest based on the fused model; and control the radiotherapy component for carrying out the radiotherapy treatment according to the plan.

It is understood that other configurations of the subject technology will become readily apparent to those skilled in the art from the following detailed description, where various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

There are shown in the drawings embodiments that are presently preferred it being understood that the invention is not limited to the arrangements and instrumentalities shown, wherein:

FIG. 1 illustrates a schematic diagram of an x-ray treatment system according to example aspects of the subject technology.

FIG. 2 illustrates an example ultrasound guided radio therapy treatment and diagnostic system according to example aspects of the subject technology.

FIG. 3 illustrates a schematic view of various components and sub-components of a radiation treatment planning (RTP) system according to example aspects of the subject technology.

### DETAILED DESCRIPTION OF THE INVENTION

The detailed description set forth below is intended as a description of various configurations of the subject technology and is not intended to represent the only configurations in which the subject technology may be practiced. The appended drawings are incorporated herein and constitute a part of the detailed description. The detailed description may include specific details for the purpose of providing a thorough understanding of the subject technology. However, the subject technology is not limited to the specific details set forth herein and may be practiced without these specific details. In some instances, structures and components are shown in block diagram form in order to avoid obscuring the concepts of the subject technology.

A hybrid image may provide accurate three-dimensional imaging for use with pathologic skin condition treatment and diagnosis including skin cancers and non-skin cancer conditions, including human based diagnostic interpretation and other means such as artificial intelligence assisted diagnosis. The hybrid image may provide diagnostic imaging, image guidance, field verification placement and localization, and treatment planning for radiation therapy and surgical interventions, such as for example Mohs surgery. Hybrid images are generated by combining imaging information acquired using different imaging technologies. For example, confocal microscopy and photoacoustic microscopy both provide structural images of a region of interest in the skin of a patient while also providing functional images of the region of interest in the skin of the patient. Confocal microscopy and photoacoustic microscopy, however, differ in the depth of penetration into the region of interest when capturing images of the region of interest. For example, when compared to each other, confocal microscopy has limited depth of penetration of up to a few hundred microns into biological samples, and photoacoustic microscopy has greater depth of penetration of up to several millimeters into biological samples. By combining the image data of the confocal microscopy and the image data of the photoacoustic microscopy, an accurate three-dimensional imaging of the region of interest can be realized resulting in accurate detection and/or diagnosis of the skin cancer or non-skin cancer condition and generation of optimal treatment plan. The detection of the skin cancer or non-skin cancer condition and generation of a treatment plan can occur in real-time. Other types of imaging may also be combined, such as computerized tomography (CT) and magnetic resonance imaging (MRI), in addition to or in the alternative to confocal microscopy and photoacoustic microscopy.

In other words, a hybrid imaging methodology disclosed herein includes a confocal microscopy imaging methodology that is optimal for imaging structural/functional features associated with a particular cutaneous lesion for a first depth, and a second imaging methodology, such as photoacoustic microscopy, that is optimal for obtaining structural/functional features associated with the cutaneous lesion for a second depth. The imaging modalities are registered and fused to form a hybrid image that combines the best features of both the confocal microscopy and photoacoustic microscopy imaging methodologies.

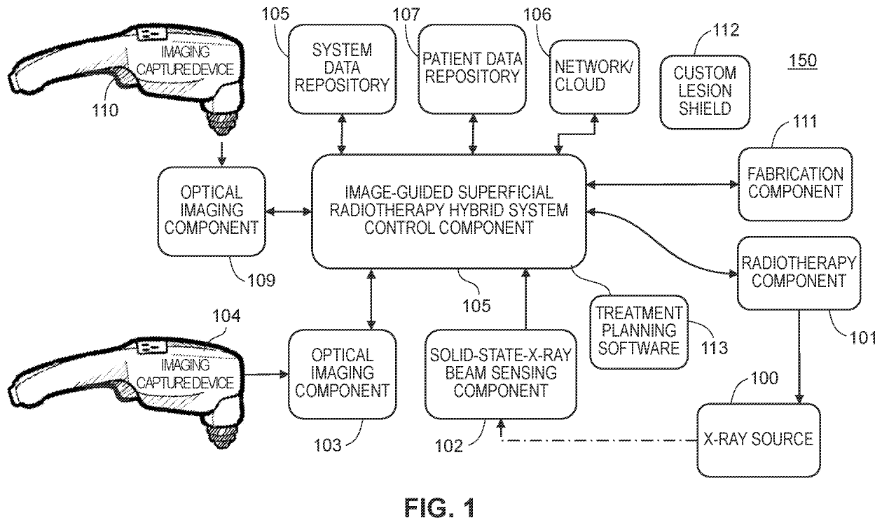

FIG. 1 illustrates a schematic diagram of a treatment system 150 according to example aspects of the subject technology. The treatment system 150 includes a radiotherapy component 101 with X-ray source 100, a solid-state X-ray beam sensing component 102, a first optical imaging component 103, and a second optical imaging component 109, and a system control component 105. The system control component 105 guides the radiotherapy component 101 based on image data obtained from the first optical imaging component 103 and the second optical imaging component 109. The first and second optical imaging components 103 and 109 communicate with the software of the system control component 105 via a bus and system drivers. The system control component 105 can also work with the solid-state X-ray beam sensing component 102 to ensure that the radiotherapy is of the appropriate intensity, depth and size. In some embodiments, the system can further include custom lesion shield 112 to protect healthy tissue from damage during treatment of a patient.

The first optical imaging component 103 includes control circuitry, system drivers, operation control software, and a first image capture device 104. According to one aspect, the first optical imaging component is a photoacoustic microscopy imaging device. Photoacoustic microscopy uses laser-induced acoustic waves to produce images of biological samples. For example, the technique of photoacoustic microscopy may involve illuminating the sample with a pulsed laser, which is absorbed by light-absorbing molecules within the sample, such as melanin. This absorption leads to localized heating and expansion creating an acoustic wave that propagates through the sample. By detecting these acoustic waves, the photoacoustic microscopy generates structural/functional image data of the sample.

The second optical imaging component 109 includes control circuitry, system drivers, operation control software, and a second image capture device 110. According to one aspect, the second optical imaging component is a confocal microscopy imaging device. Confocal microscopy involves illuminating the sample with a laser or other light source, and scanning the sample point-by-point with a focused beam of light. The light emitted by the sample is collected by a detector, and passed through a pinhole aperture to reject any light that is not in focus. Fluorescent labeling is often used in confocal microscopy to highlight specific structures within the sample to capture images of subcellular structures and molecular interactions. As is known, a biomarker (e.g., fluorescent labeling) can involve a substance which is introduced to a tissue to facilitate the identification of a disease condition such as cancer. By these techniques, the confocal microscopy generates structural/functional image data of the sample. The laser or other light source used by the second optical imaging component 109 may be the same laser or other light source used by the first optical imaging component 103. The laser or other light source may be deflected to separate lens for the first optical imaging component 103 and the second optical imaging component 109 or the laser or other light source may be modulated for the first optical imaging component 103 and the second optical imaging component 109.

The radiotherapy component 101, which can be a superficial radiotherapy component, and X-ray source 100, can together include control circuitry, one or more cooling elements for the x-ray source, power supplies, one or more high voltage generator, one or more interchangeable aluminum (Al) filter magazines, one or more collimating applicators, and one or more hardware timers that work in concert with a software timer for redundancy and other purposes.

It is contemplated that the X-ray source utilized herein will be selected so that is optimize for superficial cutaneous interaction with skin tissue, and has minimal effects at deeper tissue depths. For example a conventional superficial radiation therapy (SRT) type of X-ray unit can be used for this purpose. As will be appreciated, an SRT type of X-ray unit produces low energy X-rays that are suitable to treat skin conditions as hereinafter described.

The solid-state X-ray beam sensing component 102 can monitor the beam output of the radiotherapy component 101 and x-ray source 100, along with overall system stability and yield. The solid-state X-ray beam sensing component 102 is mounted underneath the X-Ray source 100 and is moved in front of the source when the system 150 needs to be tested for quality control, or overall system 150 diagnosis purposes. Otherwise, it is retracted back in its home position, away from the X-ray source 100 and the X-ray beam in order not to interfere during a normal operating mode.

The present disclosure contemplates that in addition to or as an alternative to using a X-ray based radiotherapy in system 150, any other types of radiotherapy can be used in system 150. Thus, the components for radiotherapy can be selected to support photon-based radiotherapy (e.g., x-rays and gamma rays), particle-based radiotherapy (e.g., electrons, protons, neutrons, carbon ions, alpha particles, and beta particles), or any combinations thereof.

A registration process is used to facilitate alignment of the image data acquired using the confocal microscopy imaging and photoacoustic microscopy imaging methods. After the region of interest has been scanned and imaged by the system 150, the image data is processed by the system's software. The image data acquired using the confocal microscopy imaging and photoacoustic microscopy imaging methods can be registered and then fused or merged to form a single image. In the fused image, the image data acquired by using the confocal microscopy imaging method is superimposed over the image data acquired by using the photoacoustic microscopy imaging method. The result is a hybrid image which includes detailed structural data for the region of interest with the functional data for the same tissue volume superimposed.

The system 150 can be used to analyze and quantify the tumor or non-skin cancer condition and subsequently prepare a treatment plan that is derived from the actual tissue parameters, such as volume, circumference, penetration depth, and tissue density. For a skin cancer application, once the tumor analysis and quantification are complete, the system 150 software provides analytical guidance to deliver the most accurate and appropriate superficial radiotherapy pertaining to the scanned and analyzed tumor. The system 150 may include treatment planning software 113 and may be operated locally or through a cloud operating system through network 106. The therapy is then delivered by the integrated superficial radiotherapy component 101. The system 150 may also delivery therapy for treatment of skin conditions, including but not limited to skin cancer, through the use of the laser in the first optical imaging component 103 and/or second optical imaging component 109. The system 150 may be delivery radiotherapy or may be a stand-alone imagining system separate from a treatment system. Additionally or alternatively, the system 150 can provide diagnostic imaging and image guidance for a surgical intervention, such as for example Mohs surgery. The system's software documents the entire diagnosis and treatment cycle and archives the patient data on a patient data repository 107 and the overall system 150 functionality log on a system data repository 108. Additionally or alternatively, the system 150 may be connected to an operating, record, and verification system that includes artificial intelligence processing capability to reduce the risk of treatment errors with use of the system 150.

The superficial radiotherapy component 101 can be utilized to treat any tumors, lesions or areas where analysis or diagnosis determines that treatment is needed. The superficial radiotherapy component 101 delivers collimated and focused x-ray photon particles to treatment areas. The system can diagnose the skin cancer or non-skin cancer condition and develop a treatment plan in real-time while the system is being used on the patient. The treatment can be without any biopsies and the pre-treatment analysis, treatment and post-treatment analysis can be carried out locally without the need for remote sources or analysis. The level of treatment can be determined as set forth below.

The system 150 is controlled and operated by the system control component 105, which can include a central computer that runs operation and control software with various parallel and connected boards that allow it to control, communicate, and monitor the various sub-components and modules of the system 150. This achieves harmonious functionality between the two main clinical components of the system 150, the superficial radiotherapy component 101, which provides radiotherapy treatment, and the first and second optical components 103 and 109, which are utilized to scan and acquire the anatomy and topology of a patient's skin area of concern for further analysis, diagnosis, quantification, and therapy planning purposes. The system control component 105 can be connected with data repositories, including a patient data repository 107 and a system data repository 108. The system 150 can also be connected to a network 106, such as a local area network, a wide area network, cloud network, and/or the Internet, which allows for clinical and system data exchange with remote systems and networks, including artificial intelligence diagnostic systems.

The system control component 105 can be configured to output a two dimensional pattern for a template or shield to be used during radiation treatment for masking or shielding certain portions of a patient's skin. The two-dimensional pattern can be output to a user in the form of an image or pattern that is suitable to facilitate manually marking and cutting a metal plate which can be used as a shield or template in accordance with a radiation therapy treatment. Alternatively, the control component 105 can output the shield pattern in a data file format which is suitable for controlling a fabrication machine. In some scenarios, a fabrication machine 111 can be included as part of the system 150. One example of a fabrication machine 111 that can be used for this purpose can include a tabletop computer numerically controlled (CNC) router (e.g., a CNC machine). However, the embodiments are not limited in this regard and the fabrication machine 111 can also comprise a 3D printer that is capable of 3D metal printing. Thereafter, the fabricated shield or template can be fabricated by the fabrication machine 111 so that it is available for use in treatment of a patient.

The patient data repository 107 and the system data repository 108 can be a solid-state drive, hard drive or other memory device. The patient data repository 107 can store patient-related data and treatment parameters, such as patient records, treatment session chronology, and disease documentation and photos. The system data repository 108 stores all system-related data and parameters, such as the system log, x-ray calibration data, and system diagnostics results. The patient data repository 107 and the system data repository 108 can be discrete devices or physically combined. One or more partitions can be used if the repositories 107 and 108 are combined, such as a single repository.

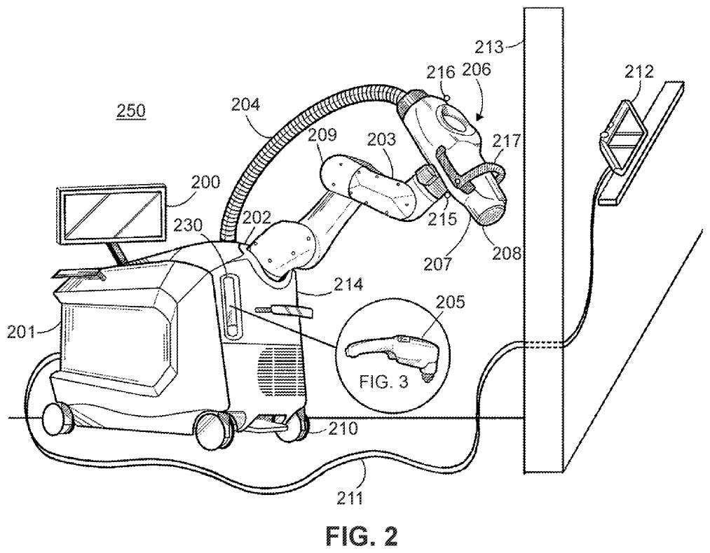

FIG. 2 illustrates an example ultrasound guided radio therapy treatment and diagnostic system 250 according to example aspects of the subject technology. The system 250 may be used for pathologic skin condition treatment and diagnosis including skin cancers and non-skin cancer conditions, including human based diagnostic interpretation and other means such as artificial intelligence assisted diagnosis. The system 250 may provide diagnostic imaging, image guidance, field verification placement and localization, and treatment planning for radiation therapy and surgical interventions, such as for example Mohs surgery. The system 250 can include a base unit 201 with various components mounted thereon or connected therewith. These components can include a radiotherapy treatment device 220 and its various components and an imaging subsystem 230.

The base unit 201 can be typically a compact unit such as one with a 30″×30″ footprint and can be mounted on casters 210 for ease of maneuverability. The base unit 201 can include a power lead for optionally providing power to all of the components housed in or connected to the base unit 201. In this regard, the base unit 201 can contain one or more computers for controlling the system 250 components and/or analyzing and processing data obtained from the system 250 components. A monitor 200 can also mounted to the base unit 201 for a user interface. Likewise, a terminal or an input device 214, such a as keyboard or mouse, can be included.

A mount 202 is provided on the base unit 201 for mounting the radiotherapy treatment device 220. The radiotherapy treatment device 220 can include a treatment arm 203 and treatment head 206, which can include removable or movable applicators 207, 208. The treatment arm 203 is articulated with appropriate retractable articulations 209. Although not shown in FIG. 2, additional articulations can also be provided at different points of system 250 to increase a number of degrees of freedom of placing and orienting treatment head 206. For example, additional articulations can be provided between treatment arm 203 and treatment head 206 and between mount 202 and treatment arm 203. Moreover, the number of articulation points illustrated in FIG. 2 is solely for ease of illustration. The present disclosure contemplates that the any number of articulation points between mount 202 and treatment head 206 can be provided so as to provide any number of degrees of freedom in treatment arm 203 required positioning and orienting the treatment head with respect to the patient.

A camera 215 can also be included to provide for remote operation or for documentation of treatment. A video-laser positioning system having camera 215 and laser or light pointer 217, which visibly marks a region with a crosshair that will receive radiotherapy treatment, can be provided. The camera 215 can capture low opacity images of the radiotherapy treatment head 206 and crosshairs of laser pointer 217 during treatment so that the exact positioning and orientation can be reproduced during subsequent treatments. In this regard, the video-laser positioning system can identify proper and precise positioning and orientation of treatment head 206. The video-laser positioning system can also allow for remote control and operation of the treatment arm 203 so that the treatment head 206 can be positioned precisely while the user is remote. In operation discussed below, the treatment arm 203 can be articulated and positioned to allow the treatment head to apply radiotherapy to a patient.

The imaging subsystem 230 can include at least one imaging head 205 attached via a corresponding lead 204 to the base unit 201 and data acquisition and processing machinery housed therein. The imaging head 205 can be a compact hand-held unit tethered to the base unit 201 by the corresponding lead 204. As such, the imaging head 205 can be freely moved to facilitate scanning different skin locations on the body of a patient. In operation, the imaging subsystem 230 can be used to collect both images and data of a diagnosis or treatment area before, during or throughout and after treatment. Additionally or alternatively, the imagining subsystem 230 may act as a therapeutic device for treatment of skin conditions, including but not limited to skin cancer, through the use of the laser, referring back to FIG. 1, in the first optical imaging component 103 and/or second optical imaging component 109. In some arrangements, an imaging head 205 can be mounted on the arm 203 instead of being provided separately.

Each imaging head can include components needed for supporting an imaging modality. For example, referring back to FIG. 1, a first imaging head 205 can be provided that includes the first optical imaging component 103 and the first image capture device 104 and a second imaging head 205 can be provided that includes second optical imaging component 109 and the second image capture device 110. However, the present disclosure also contemplates combined functionality. That is, a single imaging head 205 can incorporate the first optical imaging component 103, the first image capture device 104, the second optical imaging component 109, and the second image capture device 110.

In some embodiments, the imaging subsystem 230 may be a standalone device separate from other components of system 250, such as radiotherapy treatment device 220. The standalone imagining subsystem 230 can include one or more imaging heads 205, as described herein, such as for example, referring back to FIG. 1, a first imaging head 205 can be provided that includes the first optical imaging component 103 and the first image capture device 104 and a second imaging head 205 can be provided that includes second optical imaging component 109 and the second image capture device 110. The standalone imagining subsystem 230 can also include a single imagining head 205 with combined functionality to incorporate the first optical imaging component 103, the first image capture device 104, the second optical imaging component 109, and the second image capture device 110. The standalone imagining subsystem 230 may include various components for operation of the imagining subsystem 230, as described herein, such as computers for controlling the subsystem 230, an input device 214, and a monitor 200. The standalone imagining subsystem 230 can provide diagnostic imaging, image guidance, field verification placement and localization, and treatment planning for radiation therapy and surgical interventions and/or pathologic skin condition diagnosis including skin cancers and non-skin cancer conditions, including human based diagnostic interpretation and other means such as artificial intelligence assisted diagnosis. The standalone imagining subsystem 230 may act as a therapeutic device for treatment of skin conditions, including but not limited to skin cancer, through the use of the laser in the first optical imaging component 103 and/or second optical imaging component 109.

Lead 211 can connect the system 250 to another computer 212 or use interface that can be positioned behind a shield 213 for remote operation of the system 250 or components of system 250, such as the radiotherapy treatment device 220.

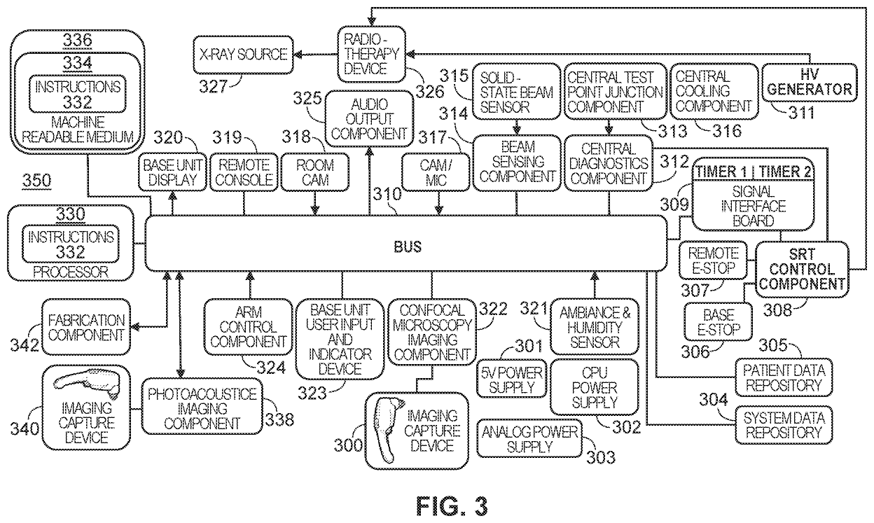

FIG. 3 illustrates a schematic view of various components and sub-components of a radiation treatment planning (RTP) system 350. The system 350 may be used for pathologic skin condition treatment and diagnosis including skin cancers and non-skin cancer conditions, including human based diagnostic interpretation and other means such as artificial intelligence assisted diagnosis. The system 350 may provide diagnostic imaging, image guidance, field verification placement and localization, and treatment planning for radiation therapy and surgical interventions, such as for example Mohs surgery. The system 350 includes a bus 310 through which the various components can communicate with each other and/or the processor 330 (e.g., a central processing unit (CPU), a graphics processing unit (GPU), or both). The processor 330 can be connected to the bus 310 as shown in FIG. 3 or integrated therewith. Power supplies 301, 302, 303 can also be included.

The system 350 can be controlled and operated by processor 330 that runs the system 350 software or instructions 332, which controls the system 350 functions, verifies the safety mechanisms, and the service and calibration functions. The processor 330 can be in communication with a machine-readable medium 334, which can be static memory 336, on which is stored one or more sets of instructions 332 (e.g., software) embodying any one or more of the methodologies or functions described herein, including those methods illustrated herein. The instructions 332 may also reside, completely or at least partially, within the system data repository 304, static memory, or within the processor 330, or a combination thereof, during execution thereof by the system 350. The system data repository and patient data repository and the processor 330 also may constitute machine-readable media.

The processor 330 can be in communication with a motherboard having an appropriate amount of static or dynamic RAM, such as 4 GB of DRAM, in order to optimally support and accommodate the operating system, main software, and real-time system monitoring functions, together with efficient patient and data system handling and archiving. The system 350 software also communicates with the peripheral components, such as Ethernet, USB, and audio/video or via network interface card in order to implement the system's user/machine interface and exchange data with external workstations and data repositories, such as electronic medical records (EMR), electronic health care records (EHR), hospital information systems (HIS), radiology information system (RIS), and picture archiving and communication systems (PACS), utilizing digital imaging and communications in medicine (DICOM) and health level 7 (HL7) communications and data structure protocols, and artificial intelligence diagnosis systems.

The system 350 can include storage mediums 304 and 305, such as solid state drives, hard drives or the like. Storage medium 304 can be the system data repository, which can include the operating system, the main system software, and system data and parameters archive. Storage medium 305 can be the patient data repository 305, which stores all patient-related data and records.

The system 350 can include a base unit that houses or otherwise provides various components of the system 350, including user interfaces. The base unit can include a base unit display device 320, such as an LCD display, and a base unit user input and indicator device 323, such as a terminal or a mouse. The system 350 can also include a remote console 319 that can be used to remotely control the system 350 so that a user does not need to be present during radiotherapy treatment. The base unit user input and indicator device 323 allows the user to interact with the system 350. The base unit user input and indicator device 323 can be utilized for initial patient data setup on the system 350 and for the ultrasound imaging of the patient's tumor at various stages of the disease before, during, and after the superficial radiotherapy period. Furthermore, the base unit user input and indicator device 323 can also be a terminal of the system 350 software. The diagnostics results and images, patient data, remote workstations topology, patient and room monitoring data, system service menus, system physics and calibration menus, and all system queues and alerts can be displayed on the base unit display device 320 or via the base unit user input and indicator device 323 as appropriate.

The system 350 can also include first and second optical imaging components 322 and 338 with first and second image capture devices 300 and 340. The first and second optical imaging components 322 and 338 each can obtain structural/functional images of a three-dimensional volume comprising a treatment area or skin lesion of concern. With the first and second optical imaging components 322 and 338 and the first and second image capture devices 300 and 340, image data representative of the treatment volume of concern can be obtained and processed.

The first optical imaging component 322 is a photoacoustic microscopy imaging device. Photoacoustic microscopy uses laser-induced acoustic waves to produce images of a treatment area or skin lesion of concern. For example, the technique of photoacoustic microscopy may involve illuminating the treatment area or skin lesion of concern with a pulsed laser, which is absorbed by light-absorbing molecules within the treatment area or skin lesion of concern, such as melanin. This absorption leads to localized heating and expansion creating an acoustic wave that propagates through the treatment area or skin lesion of concern. By detecting these acoustic waves, the photoacoustic microscopy generates structural/functional image data of the treatment area or skin lesion of concern.

The second optical imaging component 338 is a confocal microscopy imaging device. Confocal microscopy involves illuminating a treatment area or skin lesion of concern with a laser or other light source, and scanning the sample point-by-point with a focused beam of light. The light emitted by the treatment area or skin lesion of concern is collected by a detector, and passed through a pinhole aperture to reject any light that is not in focus. Fluorescent labeling is often used in confocal microscopy to highlight specific structures within the treatment area or skin lesion of concern to capture images of subcellular structures and molecular interactions. As is known, a biomarker (e.g., fluorescent labeling) can involve a substance which is introduced to a tissue to facilitate the identification of a disease condition such as cancer. By these techniques, the confocal microscopy generates structural/functional image data of the treatment area or skin lesion of concern. The laser or other light source used by the second optical imaging component 338 may be the same laser or other light source used by the first optical imaging component 322. The laser or other light source may be deflected to separate lens for the first optical imaging component 322 and the second optical imaging component 338 or the laser or other light source may be modulated for the first optical imaging component 322 and the second optical imaging component 338.

The system 350 can provide the optical imaging components 322, 338 at least partially integrated inside a housing of system 250 coupled to bus 310 with image capture devices 300, 340, outside of the housing as shown in FIG. 2. The system 350 may act as a therapeutic device for treatment of skin conditions, including but not limited to skin cancer, through the use of the laser in the optical image components 322, 338. The optical imaging components 322, 338 can also be a standalone device as described in reference to FIG. 2. The optical image components 322, 338 and other components of the system 350 can be in communication with the bus 310 and the respective other components of the system 350 utilizing interface standards such as peripheral component interconnect (PCI/PCIe), universal serial bus (USB/USBII/USBIII), or Firewire, to name a few. However, the present disclosure contemplates that any other interface and/or communications standards can be used.

The system 350 can further include a radiotherapy device 326 that includes an X-ray source 327. As discussed herein, the radiotherapy device 326 that includes an X-ray source 327 can deliver radiation therapy to a particular region or area on a patient. The radiotherapy device 326 can be coupled with a high voltage generator 311 and a central cooling component 316.

The system 350 can also include a control component, such as superficial radiotherapy control component 308, for controlling the radiotherapy provided by radiotherapy device 326. The superficial radiotherapy control component 308 can control aspects of the radiation dosage, including timing, depth and intensity. In this regard, an arm control component 324 can also be provided with the system 350 and in communication with the superficial radiotherapy control component 308 and/or processor 330. The arm control component 324 can move, articulate or otherwise control positioning of the arm to which the radiotherapy device 326 and x-ray source 327 are mounted. A base e-stop 306 and remote e-stop 307 can also be provided to provide local and remote emergency termination functions so that the radiotherapy device 326 can be stopped either locally or remotely.

Additionally, solid state beam sensing component 314 with a solid-state beam sensor 315 can be provided. In one embodiment, these components can be housed within the housing of X-ray source 327. The solid state beam sensing component 314 with a solid-state beam sensor 315 provide the ability to obtain on demand and local analysis of the radiotherapy device 326 with X-ray source 327. Utilizing the solid state beam sensing component 314 with a solid-state beam sensor 315, the radiotherapy device 326 with X-ray source 327 can be tested to determine if the radiation output is consistent with the desired radiation output. In the event that there are discrepancies, the devices can be re-calibrated or otherwise serviced.

A central diagnostics component 312 can also be provided and can be interfaced with bus 310 and processor 330. The central diagnostic component 312 is also connected with a central test point junction conjunction 313 and additionally interfaces with a signal interface board 309 that is in turn connected to both the processor 330 through bus 310 and the superficial radiotherapy control component 308. The signal interface board 309 can also include a first and second timer for redundant time counting during the application of radiation therapy, which provides for added patient safety and accurate dosimetry calculation for the delivered therapy dose to the patient. In addition to the dual hardware timers, one or more additional software based timers can be utilized or invoked by system 350.

The central diagnostics module 312 is a systems diagnostic component that monitors the various system boards and components for failures and/or errors. The central diagnostics module 312 can generate alerts regarding the system status that can either be communicated with the user, or with the system installer or manufacturer for maintenance purposes.

Additional inputs can be connected to the processor 330 through bus 310 including a camera and/or microphone 317, an audio output component 325, such as a speaker, a room camera 318 for taking pictures or video of the patient, treatment areas and/or the treatment process. An ambiance and humidity sensor 321 can also be provided in the event that conditions may affect the treatment or any of the system 350 components. However, the arrangement is not limited in this regard. A fabrication component 342 for fabrication of a metal shield or template can also be connected to the bus 310 in some embodiments.

A reference to an element in the singular is not intended to mean one and only one unless specifically so stated, but rather one or more. For example, “a” module may refer to one or more modules. An element proceeded by “a,” “an,” “the,” or “said” does not, without further constraints, preclude the existence of additional same elements.

Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

It is understood that the specific order or hierarchy of steps, operations, or processes disclosed is an illustration of exemplary approaches. Unless explicitly stated otherwise, it is understood that the specific order or hierarchy of steps, operations, or processes may be performed in different order. Some of the steps, operations, or processes may be performed simultaneously. The accompanying method claims, if any, present elements of the various steps, operations or processes in a sample order, and are not meant to be limited to the specific order or hierarchy presented. These may be performed in serial, linearly, in parallel or in different order. It may be understood that the described instructions, operations, and systems can generally be integrated together in a single software/hardware product or packaged into multiple software/hardware products.

The disclosure is provided to enable any person skilled in the art to practice the various aspects described herein. In some instances, well-known structures and components are shown in block diagram form in order to avoid obscuring the concepts of the subject technology. The disclosure provides various examples of the subject technology, and the subject technology is not limited to these examples. Various modifications to these aspects will be readily apparent to those skilled in the art, and the principles described herein may be applied to other aspects.

All structural and functional equivalents to the elements of the various aspects described throughout the disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

The title, background, brief description of the drawings, abstract, and drawings are hereby incorporated into the disclosure and are provided as illustrative examples of the disclosure, not as restrictive descriptions. It is submitted with the understanding that they will not be used to limit the scope or meaning of the claims. In addition, in the detailed description, it can be seen that the description provides illustrative examples and the various features are grouped together in various implementations for the purpose of streamlining the disclosure. The method of disclosure is not to be interpreted as reflecting an intention that the claimed subject matter requires more features than are expressly recited in each claim. Rather, as the claims reflect, inventive subject matter lies in less than all features of a single disclosed configuration or operation. The claims are hereby incorporated into the detailed description, with each claim standing on its own as a separately claimed subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirements of the applicable patent law, nor may they be interpreted in such a way.

We claim:

1. A radiotherapy method for treatment of skin, the radiotherapy method comprising:
acquiring first image data for a region of interest in the skin of a patient using a confocal microscopy;
acquiring second image data for the region of interest in the skin of the patient using a photoacoustic microscopy;
combining the first image data and the second image data to produce a fused model for the region of interest in the skin of the patient;
generating a plan for radiotherapy treatment of the region of interest based on the fused model; and
applying the radiotherapy treatment according to the generated plan.

2. The radiotherapy method of claim 1, wherein the first image data acquired using the confocal microscopy captures a first depth of the region of interest in the skin of the patient.

3. The radiotherapy method of claim 2,
wherein the second image data acquired using the photoacoustic microscopy captures a second depth of the region of interest in the skin of the patient, and
wherein the second depth is deeper than the first depth.

4. The radiotherapy method of claim 3, wherein the first depth is approximately 1 to 400 microns and wherein the second depth is approximately 0.1 to 4 millimeters.

5. The radiotherapy method of claim 1, wherein in the fused model, the first image data is superimposed over the second image data.

6. The radiotherapy method of claim 1, wherein the fused model for the region of interest in the skin of the patient includes structural data for the region of interest and functional data for the region of interest.

7. The radiotherapy method of claim 1, further comprising the step of quantifying an abnormality in the region of interest in the skin of the patient.

8. The radiotherapy method of claim 7, wherein the abnormality comprises a tumor and a non-skin cancer condition.

9. The radiotherapy method of claim 1, wherein applying the radiotherapy treatment includes use of a laser.

10. The radiotherapy method of claim 1, wherein generating the plan for radiotherapy treatment of the region of interest based on the fused model is done in real-time while the method is being used on a patient.

11. The radiotherapy method of claim 1, wherein generating the plan for radiotherapy treatment of the region of interest based on the fused model and applying the radiotherapy treatment according to the generated plan is done without a biopsy.

12. The radiotherapy method of claim 1, wherein acquiring the first image data comprises using optical imaging of the region of interest in conjunction with a use of at least one biomarker substance.

13. The radiotherapy method of claim 1, wherein generating the radiotherapy treatment plan comprises using the fused model to identify a treatment volume in the patient to which radiation is to be applied, and selecting one or more radiotherapy parameters based at least on the treatment volume.

14. The radiotherapy method of claim 1, further comprising providing a two dimensional pattern for a template or shield to be used during radiotherapy treatment for masking or shielding certain portions of a patient's skin.

15. A radiotherapy system for radiotherapy planning and treatment of skin, the radiotherapy system comprising:
a radiotherapy component comprising a radiation source configured for radiation therapy;
a first imaging component comprising a confocal microscopy configured to capture one or more optical images of a region of interest in a skin of a patient for a first depth;
a second imaging component comprising a photoacoustic microscopy configured to capture one or more optical images of a region of interest in the skin of the patient for a second depth deeper than the first depth; and
a processor configured to:
acquire, using the first imaging component, a first optical image of the region of interest for the first depth;
acquire, using the second imaging component, a second optical image of the region of interest for the second depth;
combine the first optical image and the second optical image to produce a fused model for the region of interest;
generate a plan for radiotherapy treatment of the region of interest based on the fused model; and
control the radiotherapy component for carrying out the radiotherapy treatment according to the plan.

16. The radiotherapy system of claim 15, further comprising a treatment arm and a treatment head, wherein at least the first imaging component or second imagining component is included in the treatment head.

17. The radiotherapy system of claim 15, further comprising a hand-held imaging head.

18. The radiotherapy system of claim 17, wherein the imagining head is configured to at least collect the first optical image, collect the second optical image, or provide radiotherapy treatment according to the plan.

19. The radiotherapy system of claim 17, wherein the hand-held imaging head comprises the first imaging component and second imagining component.

20. The radiotherapy system of claim 15, further comprising a first hand-held imaging head comprises the first imaging component and a second hand-held imaging head comprises the second imaging component.
